(19) United States
(12) Patent Application Publication
(10) Pub. No.: US 2013/0079602 A1
(43) Pub. Date: Mar. 28, 2013

(54) ANALYSIS OF PHYSIOLOGY BASED ON ELECTRODERMAL ACTIVITY

(71) Applicant: AFFECTIVA, INC., Waltham, MA (US)

(72) Inventors: Rosalind Wright Picard, Newtonville, MA (US); Rana el Kaliouby, Waltham, MA (US); Richard Scott Sadowsky, Sturbridge, MA (US); Oliver Orion Wilder-Smith, Holliston, MA (US)

(73) Assignee: AFFECTIVA, INC., Waltham, MA (US)

(21) Appl. No.: 13/624,894

(22) Filed: Sep. 22, 2012

Related U.S. Application Data
(60) Provisional application No. 61/538,218, filed on Sep. 23, 2011.

(51) Int. Cl. A61B 5/0205; A61B 5/053; A61B 5/08; A61B 5/01; A61B 5/024; A61B 5/00; A61B 5/11

(52) U.S. Cl. 600/301; 600/300; 600/547; 600/595; 600/549; 600/508; 600/529; 600/483; 600/484

(57) ABSTRACT

Disclosed are computer-implemented techniques for analyzing physiology based on electrodermal activity. Initially, an individual's autonomic data is captured into a computer system. The autonomic data provides information for evaluating the physiology of the individual. The autonomic data is captured through at least one sensor. Analysis, based on the autonomic data captured on the individual, is received from a web service. An output related to physiology is rendered based on the analysis which was received.

### RELATED APPLICATIONS

This application claims the benefit of U.S. provisional patent application “Analysis of Physiology Based on Electrodermal Activity” Ser. No. 61/538,218, filed Sep. 23, 2011. The foregoing application is hereby incorporated by reference in its entirety.

### FIELD OF INVENTION

This application relates generally to the analysis of physiology and more particularly to the evaluation of physiology based on electrodermal activity.

### BACKGROUND

The evaluation of an individual's physiological condition, as well as other parameters, can be extremely advantageous to the project of evaluating health and activity. Gathered physiological information may comprise a variety of parameters, including electrodermal activity (EDA)—also known as skin conductance (SC) or galvanic skin response (GSR). Such physiological information may further include skin temperature, heart rate, heart rate variability, and various other data pertaining to a human body's status. The tracking of body movement may also be beneficial, and may be collected through accelerometer readings. All these readings, as well as other information, may be collected to evaluate the health of an individual, to diagnose numerous health problems, and/or to track physical or exercise activity.

A biosensor attached to the human body may provide physiological readings and other pertinent information. Historically, however, most biosensors have been cumbersome, obtrusive, and difficult to use. The presence of such an obtrusive biosensor often impacts the user's readings simply because the wearer is cognizant of the attached biosensor. Electrodermal activity varies with multiple factors, including the moisture level of the skin. The moisture level of the skin is a function of the body's sweat glands, which are controlled by the sympathetic nervous system. Therefore, skin conductance provides an indication of psychological or physiological state. A long history of electrodermal activity research, which dates back to the 1800s, has mostly focused on the spontaneous fluctuations or reactions of electrodermal activity to stimuli. Devices for measuring electrodermal activity quantify the electrical conductance of a person's body.

### SUMMARY

Autonomic data is captured and analyzed to determine a physiological condition being experienced by a person. The autonomic data may include electrodermal activity and the analysis may be performed at or in conjunction with a web server. Analysis of autonomic data may include viewing an individual's autonomic data in light of a data set containing autonomic data of a plurality of individuals in known contexts or experiencing known physiological conditions. A computer implemented method for analyzing physiology is disclosed comprising: capturing autonomic data on an individual into a computer system wherein the autonomic data provides information for evaluating physiology of the individual and wherein the autonomic data is captured through at least one sensor; receiving analysis from a web service wherein the analysis is based on the autonomic data on the individual which was captured; and rendering an output related to physiology based on the analysis which was received.

The autonomic data may include electrodermal activity. The method may further comprise collecting accelerometer data on the individual. The method may further comprise determining contextual information based on one or more of skin temperature or the accelerometer data. The contextual information may include information for an activity that the individual is performing. The accelerometer data may be used to generate an actigraph. The method may further comprise uploading the autonomic data to the web service. The uploading may be accomplished using a mobile device. The uploading may be accomplished on an occasional basis. The method may further comprise collecting further data on one or more of heart rate, heart rate variability, accelerometer readings, respiration, or skin temperature. The sensor may be attached to an appendage of the individual. The output, which is rendered, may describe the physiology of the individual.

The method may further comprise performing signature analysis on the electrodermal activity which was collected to identify a characteristic of the electrodermal activity that corresponds to a physiological condition. The signature analysis may be based on the electrodermal activity as well as accelerometer data. The signature analysis may be based on the electrodermal activity as well as one or more of heart rate, heart rate variability, respiration, or skin temperature. The method may further comprise inferring that the individual is experiencing a physiological condition based on observing the characteristic of the autonomic data. The inferring may include a probability factor that the person is experiencing the physiological condition. The physiological condition that is being experienced may be one or more of a group including pain, anxiety, panic attack, epileptic seizure, sleep disorder, respiratory sleep problem, heart attack, depression, stress, reaction to medication, bipolar attack, distracted driving, concussion, stroke, autistic reaction, ADHD behavior, boredom, well being, being startled, being in a mood, having arthritis, cystic fibrosis, diabetes, addiction, eczema outbreak, fragile X syndrome reaction, obsessive-compulsive disorder, phobia, post-traumatic stress disorder, social anxiety disorder, or activation of sympathetic nervous system. The characteristic may be used to provide a biomarker. The signature analysis may include rise time analysis of the electrodermal activity from onset of the physiological condition. The signature analysis may include fall time analysis of the electrodermal activity from a peak value following onset of the physiological condition. The signature analysis may include time duration for elevated electrodermal activity following onset of the physiological condition. The signature analysis may include analysis of electrodermal activity peak to electrodermal activity valley range. The signature analysis may include an evaluation of a number of electrodermal activity peaks per minute. The signature analysis may include evaluation of electrodermal activity storming. The method may further comprise correlating the signature analysis to self-reporting.

The electrodermal activity may be collected with a plurality of sensors on the individual and the plurality of sensors includes at least one sensor on a left side of the individual and at least one sensor on a right side of the individual. The at least one sensor on the left side and the at least one sensor on the right side may be used to identify a difference between dominant and non-dominant electrodermal activity which was collected. The physiological condition may be analyzed as part of a clinical trial. The method may further comprise capturing autonomic data on a plurality of other people wherein the autonomic data is captured through one or more sensors. The receiving analysis from a web service may include receiving analysis related to the plurality of other people. The rendering of the output related to physiology may include a rendering about the individual. The rendering of the output related to physiology may include a rendering about the plurality of other people. The rendering may further comprise recommending a course of action based on an aggregated response of autonomic data from a plurality of other people. The method may further comprise one or more of detecting, clustering, and characterizing patterns in the autonomic data which was captured. The method may further comprise sending at least one of the autonomic data, a subset of the autonomic data, or an analysis of the autonomic data to cloud-based storage. The method may further comprise generating the analysis by the web service through cloud computation of at least one of the autonomic data, a subset of the autonomic data, or an initial analysis of the autonomic data. The analysis which is received from the web service may be based on specific access rights. The method may further comprise sending a request to the web service for the analysis. The method may further comprise, based on a signature, performing one or more of identifying when a treatment was not taken, determining treatment efficacy, analyzing time of day for treatment, identifying an adverse reaction to treatment, identifying side effects to treatment, determining treatment safety, or determining dose titration. The analysis may be generated just in time based on a request for the analysis. The method may further comprise sending a subset of the autonomic data which was captured on the individual to the web service. The data which is received may include a serialized object in a form of JavaScript Object Notation (JSON). The method may further comprise deserializing the serialized object into a form for a JavaScript object.

In embodiments, a computer-implemented method for physiology analysis may comprise: receiving autonomic data on an individual into a web-server computer system wherein the autonomic data provides information for evaluating physiology of the individual and wherein the autonomic data is captured through at least one sensor; analyzing the autonomic data on the individual which was captured; and sending an output related to analyzing that was performed. In some embodiments, a computer program product embodied in a non-transitory computer readable medium for physiology analysis may comprise: code for capturing autonomic data on an individual into a computer system wherein the autonomic data provides information for evaluating physiology of the individual and wherein the autonomic data is captured through at least one sensor; code for receiving analysis from a web service wherein the analysis is based on the autonomic data on the individual which was captured; and code for rendering an output related to physiology based on the analysis which was received. In embodiments, a computer system for physiology analysis may comprise: a memory which stores instructions; one or more processors coupled to the memory wherein the one or more processors, when executing the instructions which are stored, are configured to: capture autonomic data on an individual into a computer system wherein the autonomic data provides information for evaluating physiology of the individual and wherein the autonomic data is captured through at least one sensor; receive analysis from a web service wherein the analysis is based on the autonomic data on the individual which was captured; and render an output related to physiology based on the analysis which was received

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a system diagram for physiological analysis.

FIG. 2 is a flow diagram of physiological analysis.

FIG. 3 is a flow diagram for web services analysis.

FIG. 4 is a flow diagram for analyzing data.

FIG. 5 is a flow diagram for performing electrodermal activity (EDA) analysis.

FIG. 6 is a flow diagram of clinical trial analysis.

FIG. 7 is a system diagram on data collection.

FIG. 8 is a graphical rendering of electrodermal activity.

FIG. 9 is a graphical rendering of right and left electrodermal activity.

FIG. 10 is a graphical rendering of electrodermal activity and accelerometer data.

FIG. 11 is a graphical rendering of part of the electrodermal activity and accelerometer data.

FIG. 12 is a graphical rendering of portions of the accelerometer data.

FIG. 13 is a system diagram for mental state analysis.

### DETAILED DESCRIPTION

The present disclosure provides a description of various methods, apparatus, and systems for analysis of physiology based on electrodermal activity. Electrodermal activity reflects autonomic nervous system activity, and thus provides insight into an individual's physical or mental state. In particular, some electrodermal activity may exhibit a signature or characteristic that is associated with a physiological condition. Many such conditions are described herein, and still others will be appreciated, including pain, anxiety, panic attacks, epileptic seizures, sleep disorders, heart attacks, or the like.

By gathering data from a group of people in known contexts or experiencing known physiological conditions, it becomes possible to extract signatures in electrodermal activity data by noting correlations between the data of the group of people. Knowing these correlations allows for searching of signatures in the electrodermal activity data of an individual. When they are found in the electrodermal activity data of the individual, the physiological condition of the individual at the time the signature appeared in the electrodermal activity data may be determined or inferred. Specifically, the physiological condition of the individual at that time may be matched with a known physiological condition associated with a certain signature in the collected data from the plurality of other people.

In embodiments, the computation required to carry out the correlating, determining, inferring, and so on may occur on a client, a server, in part on a client and in part on a server, or the like. Although a variety of applications of the methods, apparatus, and systems described herein will become apparent in light of this disclosure, some applications include market research (e.g., determining visceral reactions to an advertisement or product presentation), clinical trials (e.g., determining how well or poorly an individual is reacting to a treatment or how well or poorly an individual is complying with a treatment protocol, and so on), and so forth.

Throughout this disclosure, the terms “identify” and “infer” may be used interchangeably to mean “deduce or conclude (information) from evidence and reasoning rather than from explicit statements.” Inference can include inference using probabilistic models; for example, the inference may be a 0.6 probability of a condition being present in current data. Throughout this disclosure, the phrases “other individuals” and “plurality of other people” may be used interchangeably. Throughout this disclosure, the words “signature” and “characteristic” may be used interchangeably. Although the term “biomarker” as used in the art may generally refer to a substance used as an indicator of a biological state (e.g., percent oxygenation of blood). A biomarker, as described herein, may include a physical, objective measurement of an individual, a measurement of an ability of an individual to conduct an electric signal, changes over time in that electrical signal or the ability to conduct the signal, simultaneous differences in the signal or in the ability to conduct the signal at various appendages of an individual, or the like. Throughout this disclosure, physiology may include psychophysiology. Likewise, a physiological condition may include a psycho-physiological condition, unless otherwise stated or clear from the context. It should be understood that individuals may include humans.

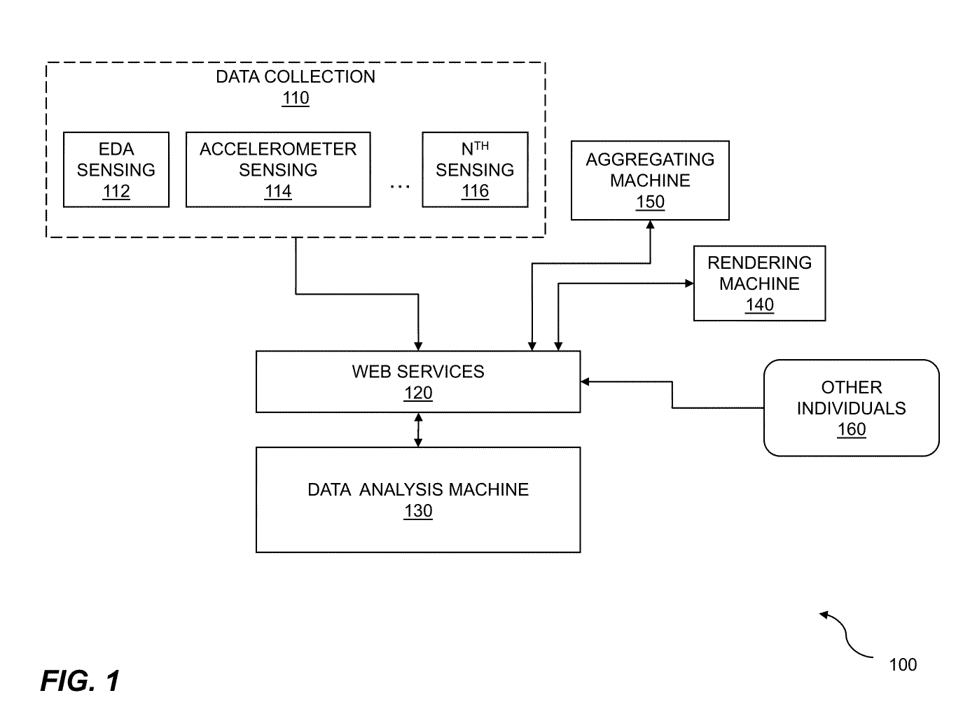

FIG. 1 is a system diagram for physiological analysis. The system 100 for physiological analysis of an individual may include data collection 110, web services 120, a data analysis machine 130, a rendering machine 140, an aggregating machine 150, and input from other individuals 160. The data collection 110 may include a plurality of sensing structures such as an EDA sensing 112, an accelerometer sensing 114, through an n^{th} sensing 116. This plurality of sensing structures may be attached to the individual, be in close proximity to the individual, or may view the individual. These sensing structures may be adapted to perform physiological analysis, which may include electrodermal activity or skin conductance (EDA sensing 112), accelerometer readings (accelerometer sensing 114), skin temperature measurements, heart rate, heart rate variability, respiration, and other types of analysis of the individual. The sensing may be done on an individual or group of people experiencing, or possibly experiencing, a physiological condition such as one or more of a group including pain, anxiety, panic attack, epileptic seizure, sleep disorder, respiratory sleep problem, heart attack, depression, stress, reaction to medication, bipolar attack, distracted driving, concussion, stroke, autistic reaction, ADHD behavior, boredom, wellbeing, being startled, being in a mood, having arthritis, cystic fibrosis, diabetes, addiction, eczema outbreak, fragile X syndrome reaction, obsessive-compulsive disorder, phobia, post-traumatic stress disorder, social anxiety disorder, or activation of sympathetic nervous system.

The data collected from these sensing structures may be analyzed in real time by the data analysis machine 130 or may be collected for later analysis, based on the processing requirements of the needed analysis. In embodiments, the analysis may be performed “just in time.” A just-in-time analysis may be performed on request—the result is provided in visual form by the rendering machine 140 when a button is selected in a web page or graphical user interface, for instance. The data analysis machine 130 may perform its analysis as data is collected so that the rendering machine 140 can present a timeline with associated analysis in real time, while the data is being collected or with little or no time lag from the collection. In this manner, the analysis results may be presented while data is still being collected on the individual. In performing its analysis, the data analysis machine may interpret the data collection 110 taken from individual in context of the data collections taken from other individuals 160. The data from the other individuals 160 may contain data collections coded with known physiological conditions, such that when patterns in the data from the other individuals 160 are correlated to the data collection 110 of the individual, a certain physiological condition of the individual may become absolutely or probably known.

The data collection 110 may include measurements from the sensing structures 112, 114, 116 taken continuously, every second, four times per second, eight times per second, thirty-two times per second, or on some other periodic basis or based on some event. The EDA sensing 112 may indicate electrodermal activity. The electrodermal activity may indicate arousal, excitement, boredom, or other mental states based on changes in skin conductance. Accelerometer sensing 114 may indicate acceleration in one, two, or three dimensions of motion. The acceleration may indicate level or type of physical activity, physical context (e.g., doing office work, riding in a car, being on a roller coaster, exercising, etc.), or the like. The various sensing means may collect any or all data needed by the data analysis machine 130 about an individual in order to perform its analysis. This data may indicate electrodermal activity, skin temperature, accelerometer readings, heart rate, respiration rate, other physiological information, or the like.

The web services 120 may support an interface and may include a server that is remote to the individual and may include cloud-based storage. Web services may include a web site, an ftp site, or a server which provides access to a larger group of analytical tools for analyzing mental states and physiological conditions. The web services 120 may also be a conduit for collected data 100 as it is routed to other parts of the system. The web services 120 may be a server or may be a distributed network of computers. The web services 120 may provide a means for a user to log in and request information and analysis. The information request may take the form of analyzing a mental state for an individual in light of various other sources of information or may be based on a group of people which correlate to the mental state for the individual of interest. In some embodiments, the web services 120 may provide for the forwarding of data which was collected to one or more processors for further analysis.

Data may be retrieved through accessing the web services 120 and requesting data which was collected for an individual. Data may also be retrieved for a collection of individuals, for a given time period, or for a given experience. Data may be queried to find matches for a specific experience, for a given mental response or mental state, or for an individual or group of individuals. Associations may be found through queries and various retrievals which may prove useful in a business or therapeutic environment. Queries may be made based on key word searches, based on time frame, or based on experience.

In embodiments, the data or analysis which is collected may be received by the web service 120 from a client device, from a server device, from a cloud device, from a virtual machine, or the like. The electrodermal activity which is collected may be communicated via a mobile device to the web service 120 for performing of a signature analysis. The electrodermal activity which is collected on the plurality of other people may be collected with a plurality of sensors on each of the plurality of other people. The plurality of sensors may include at least one sensor on a left side of each of the plurality of people and at least one sensor on a right side of each member of the plurality of people. In embodiments, the plurality of sensors may include a sensor on a left wrist and a sensor on a right wrist for each one of the plurality of people. The sensors may be placed on a right wrist region, a left wrist region, a left ankle region, and a right ankle region; a sensor on a sternum region; or the like. In embodiments, at least one sensor on the left side and at least one sensor on the right side may be used to identify a difference between dominant and non-dominant electrodermal activity which was collected.

The analysis which is received from the web service 120 may be based on specific access rights. For example, a machine receiving the analysis may be authenticated and granted access to the analysis based upon business rules. For another example, a user name and password may be provided to the web service 120 and the web service 120 may validate the user name and password prior to the web service 120 transmitting the analysis. By way of example, and not of limitation, a clinical director may be able to view aggregated responses and/or clusters of information while an individual may only be able to see their own personal data. A variety of examples of access rights and ways of enforcing access rights will be appreciated.

In embodiments, the web services 120 may forward data to the data analysis machine 130. The data analysis machine 130 may include any suitable computer or virtual machine adapted, at minimum, to receive electrodermal sensing 112 data (or aggregate statistics thereof) on a plurality of people, to receive electrodermal sensing 112 data indicating electrodermal activity of an individual, and to identify at least a characteristic of the electrodermal activity of the individual in view of the electrodermal sensing 112 data (or aggregate statistics thereof) on the plurality of people. A characteristic of the electrodermal activity may be some difference after onset of physiological change such as changing tonic levels to baselines and norms from the individual and from others. The characteristic may include the number of peaks per minute, rise time, fall time, elevated time duration, bilateral difference, difference between dominant and non-dominant sides, peak to valley range, storming activity, or the like. In embodiments, accelerometer data may be collected on the plurality of people and a characteristic of the accelerometer data may be identified and used with the characteristic of the electrodermal activity. The characteristic of the electrodermal activity and the characteristic of the accelerometer data may each be used for inferring physiological condition experiences. The data analysis machine 130 may perform signature analysis on the electrodermal activity of the electrodermal sensing 112 data to identify the characteristic of the electrodermal activity of the individual.

In embodiments, a plurality of people may be experiencing a particular physiological condition, so the electrodermal sensing 112 data (or aggregate statistics thereof) on the plurality of people may correspond to that specific physiological condition. The data analysis machine 130 may perform signature analysis on this electrodermal sensing 112 data to identify a characteristic of electrodermal activity that corresponds to this physiological condition. The data analysis machine 130 may then collect the electrodermal sensing 112 data indicating electrodermal activity of the individual and look for the identified characteristic. From this, the data analysis machine 130 may infer that the individual is experiencing the physiological condition based on observing the characteristic of the electrodermal activity on the individual which was collected.

In embodiments, the plurality of people may be part of a clinical trial, so the data analysis machine 130 may enable use of the characteristic of the electrodermal activity as an objective biomarker during a clinical trial. The objective biomarker may be used to characterize a difference between a control group and a treated group, evaluate different responses from differing demographics, evaluate when treatment was not taken, determine treatment efficacy, identify an adverse reaction, identify side effects, determine treatment safety, determine dose titration, or the like.

In some embodiments, a depiction is provided using a rendering machine 140. The rendering machine 140 may be part of the web services 120, may be part of a client computer system, or the like, and may include a display device on which the depiction is provided. The rendering machine 140 may produce the depiction of information collected in the data collection 110. The depiction may include display of video, electrodermal activity, accelerometer readings, skin temperature, heart rate, heart rate variability, a malady indication, a probability of a malady, or the like. The depiction may also include display of mental states. In some embodiments, the depiction may include probabilities of certain mental states.

The physiological condition for the individual may be inferred based on the data which was collected and may be based on electrodermal activity. For instance, an elevated heart rate may indicate being excited. Reduced skin conductance may correspond to arousal. These and other factors may be used to identify mental states that the rendering machine 140 depicts on its display device.

The aggregating machine 150 may analyze other data to aid in the evaluation of the physiological condition of the individual. The other data may include various information about the other individuals on whom sensing was performed, including demographics or other treatment experiences. The other data may also include further medical information which might be relevant to clinical trials. The other data may also include various drug sensitivities of the people participating in the test. The other data may include data collected from the other individuals 160. This data from the other individuals 160 may contain output from the data collection 110 coded with known physiological conditions. As part of analyzing the other data to aid in the valuation of the physiological condition of the individual, the aggregating machine 150 may produce aggregate statistics (or the like) from the other data. Embodiments of the aggregating machine may include a computer, virtual computer, or the like. The other individuals 160 may be selected according to a random sampling technique, may be participants in a clinical trial, may be self-selected, may be members of a cohort, and so on.

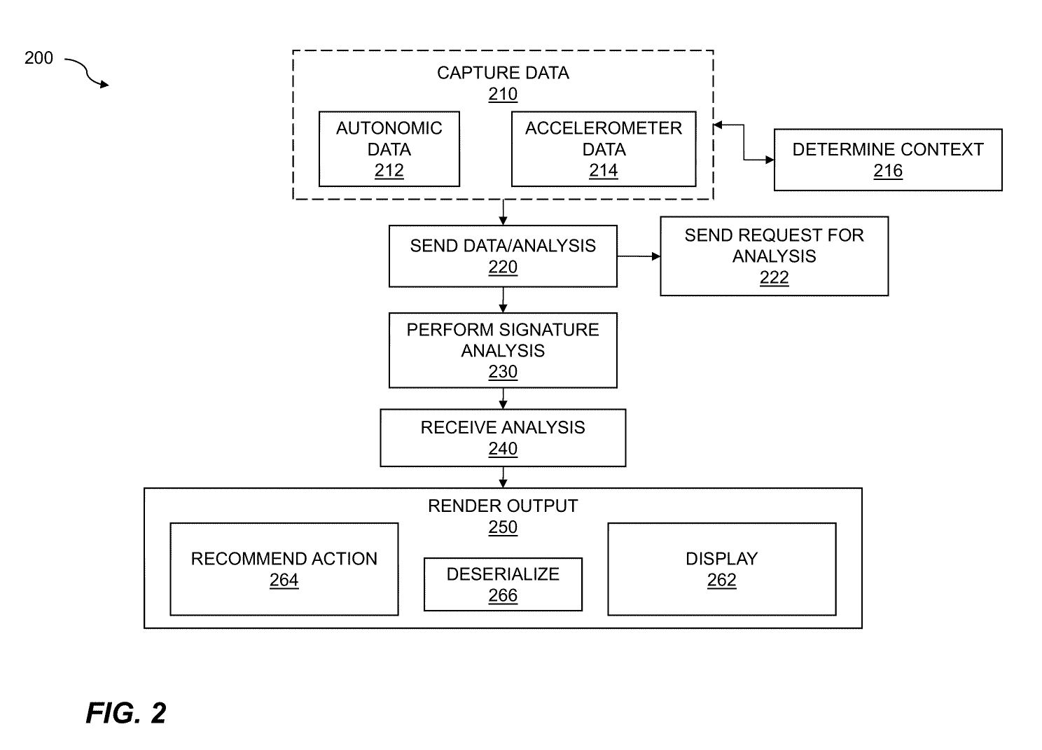

FIG. 2 is a flow diagram of physiological analysis. The flow 200 depicts a computer implemented method for analyzing autonomic and accelerometer data to produce an analysis of the physiology of an individual. The flow 200 may include capturing data 210 (such as autonomic data 212, accelerometer data 214, or the like) on an individual into a computer system wherein the autonomic data 212 provides information for evaluating the physiology of the individual and wherein the autonomic data 212 is captured through at least one sensor. In some embodiments, the sensor or sensors may be attached to one or more appendages of the individual. The autonomic data 212 may include electrodermal activity and may be collected by an EDA sensing structure 112. The flow 200 may include collecting accelerometer data on the individual which may be collected while the individual is experiencing a physiological condition. Physiological conditions may include pain, anxiety, panic attacks, epileptic seizures, sleep disorders, heart attacks, depression, stress, reaction to medication, bipolar attack, distracted driving, concussion, stroke, autistic reaction, ADHD behavior, boredom, wellbeing, being startled, being in a mood, having arthritis, cystic fibrosis, diabetes, addiction, eczema, fragile X syndrome, obsessive-compulsive disorder, phobias, post-traumatic stress disorder, social anxiety disorder, activation of sympathetic nervous system, or the like. The accelerometer data 214 may be captured via an accelerometer sensing structure 114. The flow 200 may include collecting accelerometer data from the plurality of other people and the individual. In some embodiments, the accelerometer data may be considered actigraphy data and the accelerometer data may be used to generate an actigraph. The accelerometer data 214 may contextualize an activity that the individual is performing. For example, and without limitation, the accelerometer data 214 may indicate hand or wrist movements consistent with the activity of typing, or it may indicate no movement at all, which may be consistent with sitting still or sleeping. Continuing, the accelerometer data may indicate accelerating and decelerating movement consistent with driving in stop-and-go traffic, and so on. In embodiments, capturing data 210 may include calculating an initial analysis of at least the autonomic data 212. Such initial analysis may include pre-processing the autonomic data 212 to remove anomalies, to remove data that is irrelevant or untrusted, or the like. For example and without limitation, when interested in the electrodermal activity of a test user at rest, the autonomic data 212 taken during periods of high activity as indicated by the accelerometer data 214 may be discarded as unreliable due the an increased likelihood that perspiration from the high activity may result in anomalous reading of the autonomic data 212. The flow 200 may include determining contextual information 216 based on one or more of the skin temperature or accelerometer data. The contextual information may include information for an activity that the individual is performing.

The flow 200 may continue with sending the data or initial analysis 220 which was captured to another system and/or sending a request to the web service for an analysis 222 of the data which was captured 200. The process may include uploading the autonomic data to the web service. The uploading may be accomplished using a mobile device. The uploading may be accomplished on an occasional basis. The occasional basis may be periodic, when a buffer is full, when wireless coverage is available, when an event of interest occurs, or the like. The sending may include sending at least one of the autonomic data, a subset of the autonomic data, an analysis of the autonomic data, or the like to cloud-based storage.

In embodiments, sending the data to another system may include sending one of the autonomic data 212, a subset of the autonomic data 212, and an initial analysis of the autonomic data 212 to a cloud-based storage. In embodiments, sending the data to another system may include sending a subset of the autonomic data 212 which was captured on the individual to the web service 120. In embodiments, sending the request for analysis may include sending a request to the web service 120 for the analysis. The analysis may be generated just in time based on the request for the analysis. In embodiments sending the data or sending the request may include sending the data or request from a client to the web service 120, through the web service 120 to the data analysis machine 130, through the web service 120 to the aggregating machine 150, or the like. Without limitation, in embodiments a sender of the data or request may be a client and the receiver may be a server.

The flow 200 may continue with performing a signature analysis 230 based on the autonomic data 212 captured on the individual. In embodiments, the web service 120 may generate the analysis through cloud computation of at least one of the autonomic data 212, a subset of the autonomic data 212, and an initial analysis of the autonomic data 212. The signature analysis 230 may be automatic and may be done on the autonomic data 212 which was collected in order to identify a characteristic of the autonomic data that corresponds to a physiological condition. In embodiments the physiological condition may be studied as part of a clinical trial.

In embodiments, performing the signature analysis 230 may include at least one of detecting, clustering, and characterizing patterns in the autonomic data which was captured. In embodiments, performing the signature analysis 230 may include aggregating further autonomic data 212 from a plurality of other people with the autonomic data 212 of the individual. In some embodiments, the aggregating may include deriving norms based the plurality of other people and comparing the autonomic data of the individual with the norms that were determined. In embodiments, performing the signature analysis 230 may include correlating one of the autonomic data 212, a subset of the autonomic data, and an initial analysis of the autonomic data with a physiological condition.

The flow 200 may continue with receiving analysis 240 from a web service 120 wherein the analysis is based on the autonomic data 212 on the individual which was captured. The receiving of analysis 240 may include receiving analysis related to the plurality of other people as well as analysis information is related to autonomic data 212. Thus, the analysis which was received from the web service 120 may be based on further autonomic data. The further autonomic data may include one or more of heart rate, heart rate variability, respiration, or skin temperature. By way of example and without limitation, analysis of autonomic data 212 from a crowd of people watching a movie may be provided in tandem with the analysis of autonomic data 212 of the individual who may also be watching the movie. In this way, the individual's reaction to scenes in the movie may be compared with those of the crowd. A variety of other examples will be appreciated. Further, the analysis which was received from the web service may be based on a correlation between one or more of the electrodermal activity data, a subset of the electrodermal activity data, and an analysis of the further autonomic data with a physiological condition. The analysis may include a correlation for the physiology of the plurality of other people to the physiology of the individual. The correlation may be based on metadata from the individual and metadata from the plurality of other people.

In embodiments, the analysis which was received from the web service 120 may be based on a correlation between one of the autonomic data 212, a subset of the autonomic data, and an initial analysis of the autonomic data 212 with a physical condition. The analysis may include a correlation for the physiology of a plurality of other people to the autonomic data 212 which was captured on the physiology of the individual. In embodiments, the correlation may be based on metadata from the individual and metadata from the plurality of other people.

The flow 200 may continue with rendering output 250 related to the physiology based on the analysis which was received from the web service 120. The rendering of the output related to physiology may include a rendering about the individual. The output, which is rendered, may describe the physiology of the individual. The rendering of the output related to physiology may include a rendering about the plurality of other people. In embodiments, the rendering machine 140 may render the output. The output which is rendered may include graphical or textual display 262 of information collected in the data collection 110. The display 262 may include display of electrodermal activity, accelerometer readings, skin temperature, heart rate, heart rate variability, or the like. The display 262 may describe the physiology or physiological condition of the individual. The physiological condition for the individual may be inferred based on the data which was collected and may be based on analysis of electrodermal activity. The rendering may further comprise recommending a course of action 264 based on the physiology of the individual. For example, the recommendation may include one or more of a group comprising modifying a titration level, modifying a cognitive behavioral therapy program, modifying an occupational therapy program, modifying pain management procedures, changing a medical consultation presentation, and changing of customer service. The recommending a course of action 265 may be based on an aggregated response of autonomic data from a plurality of other people. The rendering may further comprise recommending a course of action 264 based on an aggregated response of the autonomic data 212 captured from the plurality of other people. By way of example, and not of limitation, a drug may be removed from trial based on a side effect observed on a plurality of other people; the side effect may be measured using electrodermal activity or other autonomic data. The plurality of other people may be a subset of a population and may be correlated to a certain demographic. The output which is rendered may be based on data which is received from the web service 120. In embodiments, the data which is received may include a serialized object in a form of JavaScript Object Notation (JSON), and rendering output 250 may further include deserializing 266 the serialized object into a form for a JavaScript object. Various steps in the flow 200 may be changed in order, repeated, omitted, or the like without departing from the disclosed inventive concepts. Various embodiments of the flow 200 may be included in a computer program product embodied in a non-transitory computer readable medium that includes code executable by one or more processors.

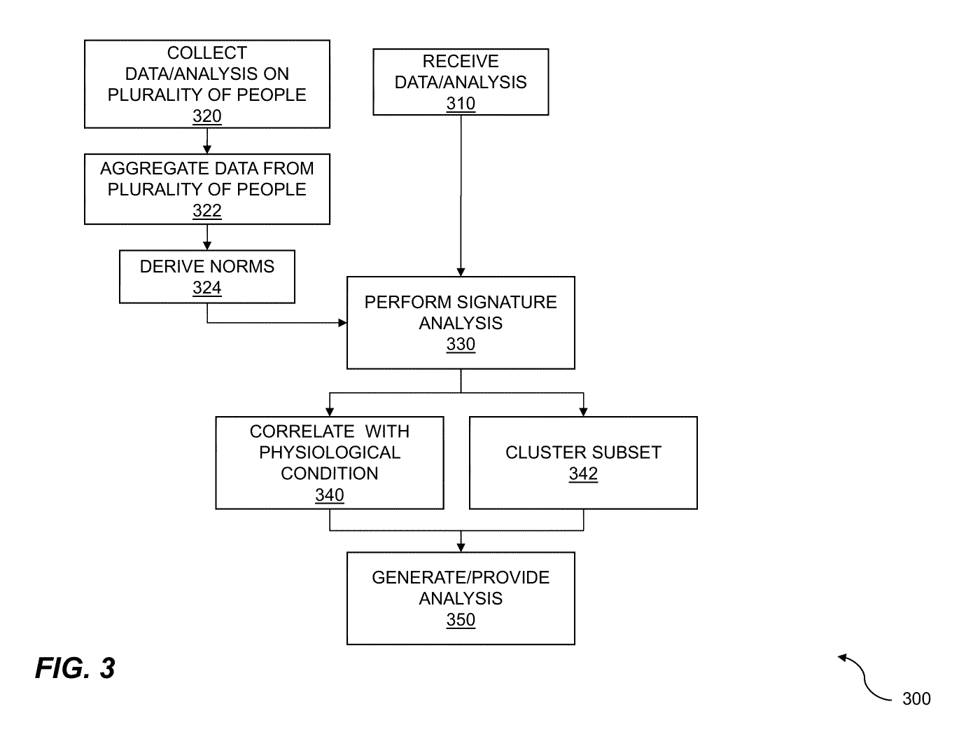

FIG. 3 is a flow diagram of web services analysis. The flow 300 describes a computer implemented method for analyzing autonomic and accelerometer data using a web service 120 to produce an analysis of the physiology of an individual or individuals. The flow 300 may include collecting data or analysis such as electrodermal activity on an individual or an analysis of electrodermal activity on an individual and receiving that data or analysis 310. The flow 300 may also include capturing autonomic data on a plurality of other people 320 wherein the autonomic data from the plurality of other people is captured through one or more sensors and wherein the plurality of people are experiencing a physiological condition. The autonomic data may include electrodermal activity data. Further, the flow 300 may continue with aggregating the electrodermal activity data from the plurality of other people 322 with the electrodermal activity of the individual. The aggregating may include deriving norms 324 based the plurality of other people and comparing the electrodermal activity data of the individual with the norms that were determined. In embodiments, the data which is collected may, in addition to electrodermal activity, include further data such as a heart rate, heart rate variability, accelerometer readings, respiration, skin temperature, or the like. The further data may be used with the collected electrodermal activity to provide context for a signature analysis, such as described hereinafter and elsewhere.

The flow 300 may continue with performing a signature analysis 330 on the data—for example, electrodermal activity—which was collected in order to identify a characteristic of the data that corresponds to a physiological condition. Such identifying or inferring may include using a probability factor to determine whether or not the individual is experiencing a certain physiological condition experienced by the plurality of people. Signature analysis may include identifying an asymmetry in electrodermal activity between the left side and the right side, which may then be used as evidence in identifying or inferring the characteristic of the data that corresponds to the physiological condition. The signature analysis may include analysis of the rise time of the electrodermal activity from the onset of the physiological condition. The signature analysis may also include analyzing the fall time of the electrodermal activity from a peak value following onset of the physiological condition. The signature analysis may also include analysis of electrodermal activity peak to electrodermal activity value range. The signature analysis may include evaluation of the number of electrodermal activity peaks per minute. The signature analysis may include evaluation of electrodermal activity storming. The signature analysis may provide an objective biomarker for the physiological condition.

The flow 300 may continue with correlating one or more of the electrodermal activity data, a subset of the electrodermal activity data, an analysis of the electrodermal activity data, accelerometer data, or an analysis of the further autonomic data with a physiological condition 340. The correlating may include using a multidimensional set of variables and linearly or nonlinearly mapping of these variables to one or more outcome self-reported measures. The correlating may be used with machine learning methods, prediction methods, regression analysis, or the like. In one example spectra of the electrodermal activity may be computed and compared with the self-reported pain data. The comparison may include a squared coherence between the two. Where the squared coherence is significantly higher than a random noise comparison, the frequency range where it was maximized may be identified. That range then may be used to process the electrodermal activity to find where it is most similar to the self-report measure.

The flow 300 may include clustering a subset 342 of the plurality of other people based on a signature which is identified during signature analysis. The subset may correspond substantially to one of a control group, a treated group, and a portion of the treated group. The subset may be identified as part of an adaptive trial. The subset may correspond to a demographic within the plurality of other people. The flow 300 may continue with generating or providing an analysis of the data or the analysis which was received 350. In embodiments, generating or providing the analysis may include transmitting the analysis from the web service 120 to a client computer or other computer. Various steps in the flow 300 may be changed in order, repeated, omitted, or the like without departing from the disclosed inventive concepts. Various embodiments of the flow 300 may be included in a computer program product embodied in a non-transitory computer readable medium that includes code executable by one or more processors.

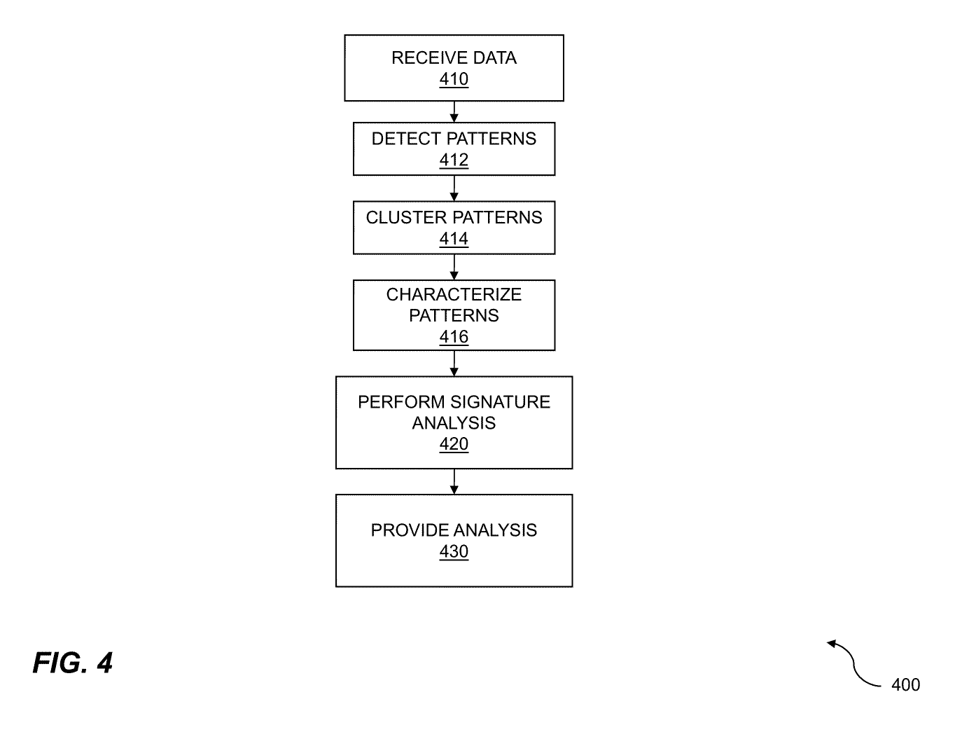

FIG. 4 is a flow diagram for analyzing data. The flow 400 describes a computer implemented method of analyzing electrodermal activity data. Data including at least electrodermal activity data is received 410. In embodiments the data may be received by a processing device such as a client, a server, a cloud service, or the like. The flow 400 may continue with detecting patterns in the data 412, analyzing cluster patterns in the data 414, and characterizing the patterns 416 in the electrodermal activity data which was captured. Characterization of the patterns may include associating the patterns from data of an individual with patterns from data of a plurality of other individuals. In embodiments, clusters of patterns from the data of the plurality of other individuals may be associated with physiological conditions, so that the clustered patterns in the data of the individual may be identified or inferred as indicating the physiological conditions in the individual at the time the individual exhibited the electrodermal activity encoded in the received data. The flow 400 may include performing signature analysis 420 on the electrodermal activity, or other data, which was collected to identify a characteristic of the electrodermal activity that corresponds to a physiological condition. The signature analysis may be based on the electrodermal activity as well as accelerometer data and may include identifying a combination of certain electrodermal activity along with certain accelerometer data. The signature analysis may be based on the electrodermal activity as well as one or more of heart rate, heart rate variability, respiration, or skin temperature.

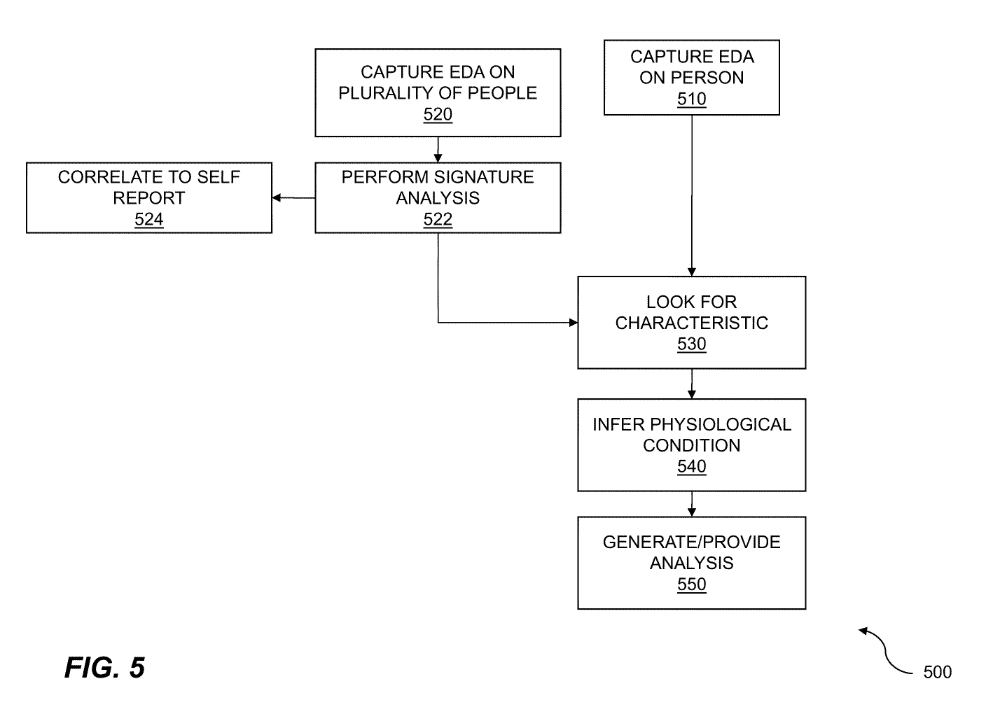

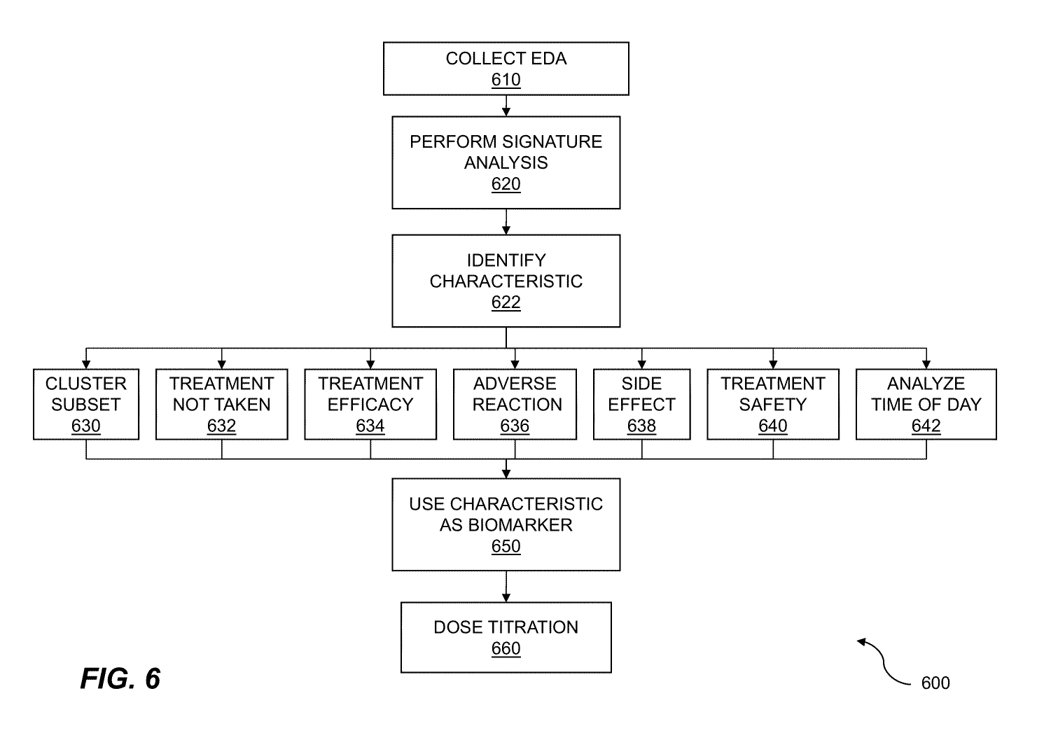

The flow 400 may continue with performing a signature analysis on detected, clustered, and characterized patterns. Signature analysis is described in greater detail hereinafter with reference to FIG. 5, FIG. 6, and elsewhere. The flow 400 may continue with providing output of the signature analysis 430, such as by transmitting the output to a remote system, saving the output to disk, displaying the output on a display device, or the like. Various steps in the flow 400 may be changed in order, repeated, omitted, or the like without departing from the disclosed inventive concepts. Various embodiments of the flow 400 may be included in a computer program product embodied in a non-transitory computer readable medium that includes code executable by one or more processors.

FIG. 5 is a flow diagram for performing electrodermal data analysis. The flow 500 describes a computer implemented method for inferring a physiological condition based at least upon electrodermal activity. The flow 500 may include capturing electrodermal activity data on an individual 510 into a computer system wherein the electrodermal activity data provides information on physiology of the individual and wherein the electrodermal activity data is captured through a sensor. The flow 500 may include capturing electrodermal data on a plurality of other people 520. The flow 500 may include performing signature analysis 522 on the electrodermal activity data. This signature analysis may identify signatures in the data that correlate across at least some of the plurality of other people. The flow 500 may include correlating the signature analysis to self-reporting 524 where the self-reporting includes reports of physiological conditions provided by the plurality of other individuals during the collection of electrodermal activity data on them. For example, the plurality of other people may experience anxiety. A trigger, such as watching a suspenseful movie, may be used in the evaluation of anxiety. During a particular sequence in the movie, the electrodermal activity of each of the plurality of other people may trend more or less in unison as the plurality of other people experience an autonomic response (e.g., feelings of fear) at the same time. Either at that time or later, when asked to recall the sequence, each of the plurality of people may self report that the movie sequence was, for example, scary or anxiety causing. The self-reporting may be done with paper and pencil, turning a dial, inputting data to a digital mobile device, or the like. Having identified the trend or signature in the electrodermal activity by analysis, and having received self reports from the plurality of people exhibiting evidence of feeling scared at the time that the trend or signature appeared in the electrodermal activity, that trend or signature in electrodermal activity may be inferred to be correlated to the feeling of fear. The flow 500 may continue to look for that signature or characteristic 530 in the electrodermal activity data of the individual. Context may be analyzed to help in identification of the characteristic 530. In embodiments, context may be identified based on other sensors such as accelerometers, skin thermometers, or the like. In some embodiments, the context may provide greater confidence that a characteristic is identified. A signature may be a combination of characteristics of multiple sensor readings. By way of example, cocaine usage may be indicated by elevated electrodermal activity along with lowered skin temperature and sustained increased motion. Certain time constants and typical morphologies may be associated with the characteristics. Having found a signature, the flow 500 may include inferring that the individual is experiencing a physiological condition based on observing the characteristic of the autonomic data. The inferring may include a probability factor that a person is experiencing the physiological condition.

The flow 500 may continue to generate or provide an analysis 550 that may include statistics or identities of the individual and the plurality of other people, the trend or signature in electrodermal activity identified in the received data, the inferred physiological condition of the individual, or the like. In embodiments, generating or providing the analysis may include transmitting it, saving it, displaying it, or the like. Various steps in the flow 500 may be changed in order, repeated, omitted, or the like without departing from the disclosed inventive concepts. Various embodiments of the flow 500 may be included in a computer program product embodied in a non-transitory computer readable medium that includes code executable by one or more processors.

FIG. 6 is a flow diagram of clinical trial analysis. The flow 600 may describe evaluating a physiological condition that is analyzed as part of a clinical trial. The flow 600 may include collecting data such as electrodermal activity 610 on a plurality of people who are part of a clinical trial. Collecting data 610 may further include collecting accelerometer data on the plurality of people who are part of the clinical trial. Collecting data 610 may further include collecting skin temperature data on the plurality of people who are part of the clinical trial. The flow 600 may continue with performing signature analysis 620 on the electrodermal activity which was collected to identify a characteristic of the electrodermal activity. The signature analysis 620 may include rise time analysis of the electrodermal activity from onset of the physiological condition. The signature analysis 620 may include fall time analysis of the electrodermal activity from a peak value following onset of the physiological condition. The signature analysis 620 may include time duration analysis for elevated electrodermal activity following onset of the physiological condition. In some embodiments, the signature analysis may be augmented by facial data collected from individuals. The flow 600 continues with identifying a characteristic 622 based on the signature analysis where the characteristic may provide an objective biomarker.

Based upon the analysis, a number of things may be identified or determined. The flow 600 may continue with clustering a subset 630 of the plurality of people based on a signature which is identified during the signature analysis. This subset may correspond substantially to one of a control group and a treated group; to a demographic within the plurality of people; or the like. The flow 600 may continue with identifying when a treatment was not taken 632 by an individual based on a signature that was identified during the signature analysis. The flow 600 may continue with determining treatment efficacy 634 based on a signature that was identified during the signature analysis. The flow 600 may continue with identifying an adverse reaction 636 to treatment based on a signature that was identified during the signature analysis. The flow 600 may continue with identifying a side effect 638 to treatment based on a signature that was identified during the signature analysis. The flow 600 may continue with determining treatment safety 640 based on a signature identified during the signature analysis. The flow 600 may include analyzing time of day 642 (chronobiological) treatment based on a signature identified during the signature analysis. The signature analysis may include time duration analysis for elevated electrodermal activity following the onset of the physiological condition. The signature analysis may include analysis of electrodermal activity peak to electrodermal activity valley range. The signature analysis may include an evaluation of the number of electrodermal activity peaks per minute. The signature analysis may include evaluation of electrodermal activity storming.

The flow 600 may continue by using the characteristic to provide a biomarker 650. The flow 600 may include determining dose titration 660 based on a signature identified during the signature analysis. In embodiments, a dose titration 660 may be determined based on a characteristic used as a biomarker. The flow 600 may include, based on a signature, performing one or more of identifying when a treatment was not taken, determining treatment efficacy, analyzing time of day for treatment, identifying an adverse reaction to treatment, identifying side effects to treatment, determining treatment safety, or determining dose titration.

Various steps in the flow 600 may be changed in order, repeated, omitted, or the like without departing from the disclosed inventive concepts. Various embodiments of the flow 600 may be included in a computer program product embodied in a non-transitory computer readable medium that includes code executable by one or more processors.

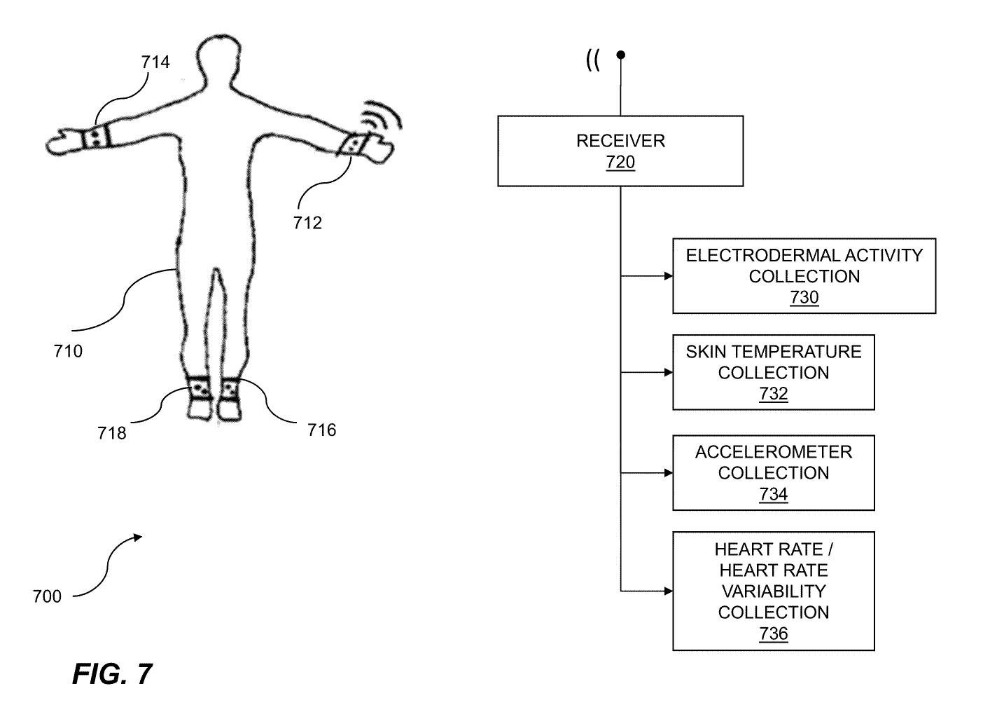

FIG. 7 is a diagram on data collection. The diagram 700 shows an instrumented individual and a system for data collection. The instrumented individual 710 is wearing a right wrist sensor 712, a left wrist sensor 714, a right ankle sensor 716, and a left ankle sensor 718. Each of the sensors 712, 714, 716, and 718, may include detectors for electrodermal activity, skin temperature, acceleration, heart rate, blood pressure, other physiological factors, other physical factors, or the like. In embodiments, electrodermal activity may be collected with a plurality of sensors on the individual and the plurality of sensors may include at least one sensor on a left side of the individual and at least one sensor on a right side of the individual. The at least one sensor on the left side and the at least one sensor on the right side may be used to identify a difference between dominant and non-dominant electrodermal activity which was collected.

Each of these sensors may transmit information collected to a receiver 720 using wireless technology such as IEEE 802.11x, Bluetooth, cellular, or the like. In embodiments, each of these sensors may store data and burst download the data through wireless technology. In embodiments, each of these sensors may store information for later wired download. The receiver 720 may provide data received from these sensors to one or more components of a system. Providing the data may occur periodically, sporadically, continuously, or the like. The one or more components of the system may include an electrodermal activity collection structure 730, a skin temperature collection structure 732, an accelerometer collection structure 734, a heart rate/heart rate variability collection structure 736, or the like. Embodiments of each of these structures may be implemented in hardware, software, a combination of hardware and software, or the like. Embodiments of each of these structures may receive data corresponding to two through four of the sensors 712, 714, 716, and 718, and may be able to track differences between any two of them. In embodiments, these sensors may be attached to the individual in pairs, with one sensor of a pair on a left appendage and the other sensor of the pair on a right appendage. The left and right appendages could be palms, hands, wrists, forearms, elbows, arms, feet, ankles, legs, knees, thighs, or the like. Sensors could also be placed on the sternum, head, or elsewhere.

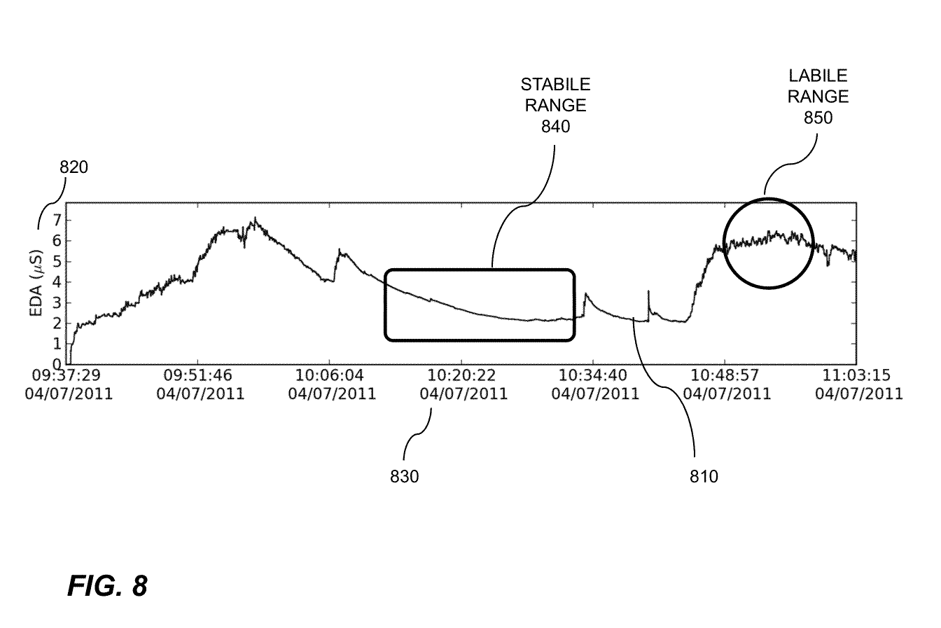

FIG. 8 is a graphical rendering of electrodermal activity. The rendering shows a plot of electrodermal data 810 measured in micro-siemens 820 over time 830. The rendering shows a stabile range 840 with a smooth trend over time, and a labile range 850 with numerous peaks and troughs over time. The stabile range 840 and the labile range 850 may represent signatures in the electrodermal activity of an individual or person, as described above and elsewhere. The labile region may be evaluated for the number of electrodermal activity peaks per minute. In some embodiments, numbers such as two, three, four, five, or six peaks per minute, and clusters of adjacent or nearly adjacent regions that meet such peak frequency criteria, may be used to delineate electrodermal activity storming.

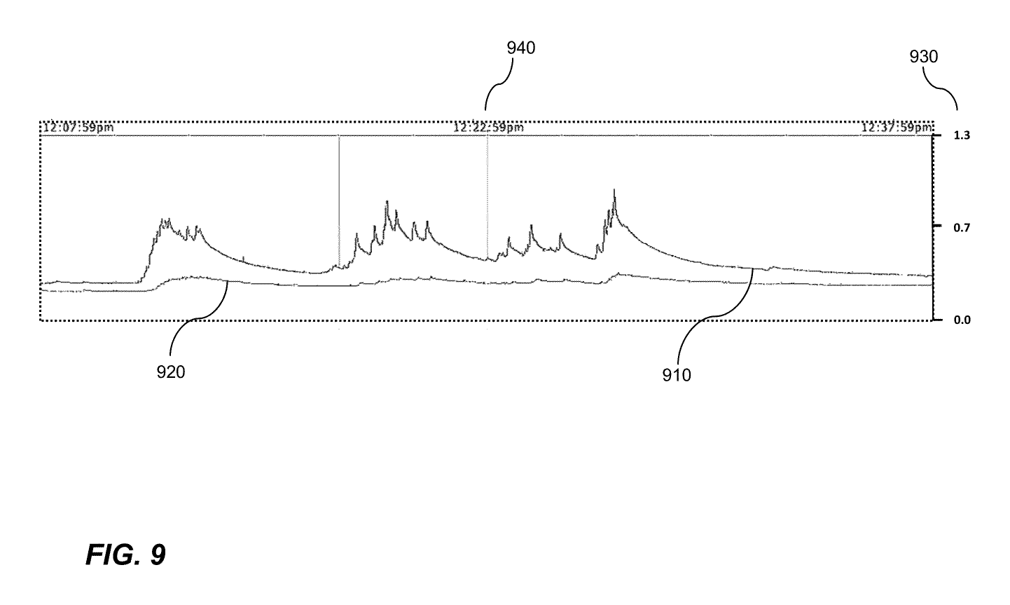

FIG. 9 is a graphical rendering of right and left electrodermal activity. Curves plotting the left electrodermal activity 910 and the right electrodermal activity 920 may be measured in micro-siemens 930 and are shown over time 940. The difference between the activities 910 and 920 can be clearly seen and may represent signatures in the electrodermal activity of an individual or person, as described above and elsewhere. Signatures or features of interest may include areas under the curves, percentage of time that one curve is less than the other during inactive moments of wake or sleep, relative number of peaks per minute within a region of interest, or the like. In embodiments, readings from an accelerometer may be used to determine when an individual is awake or asleep, what segment of time constitutes a region of interest, and so on. For example, times when the accelerometer measures no change in motion, or changes in motion below a certain threshold, might indicate that the individual is asleep. In embodiments, electrodermal activity and accelerometer measures may be evaluated in tandem, even when active motion is neither anticipated nor a primary concern. In some embodiments, the electrodermal activity curves may be smoothed and a correlation between the two may be calculated. The magnitude of this correlation may be compared relative to both activity level and times of day that might be most associated with a physiological condition of interest. In this way, it may be determined whether an individual is likely to have been experiencing the condition of interest at the time the activities were recorded.

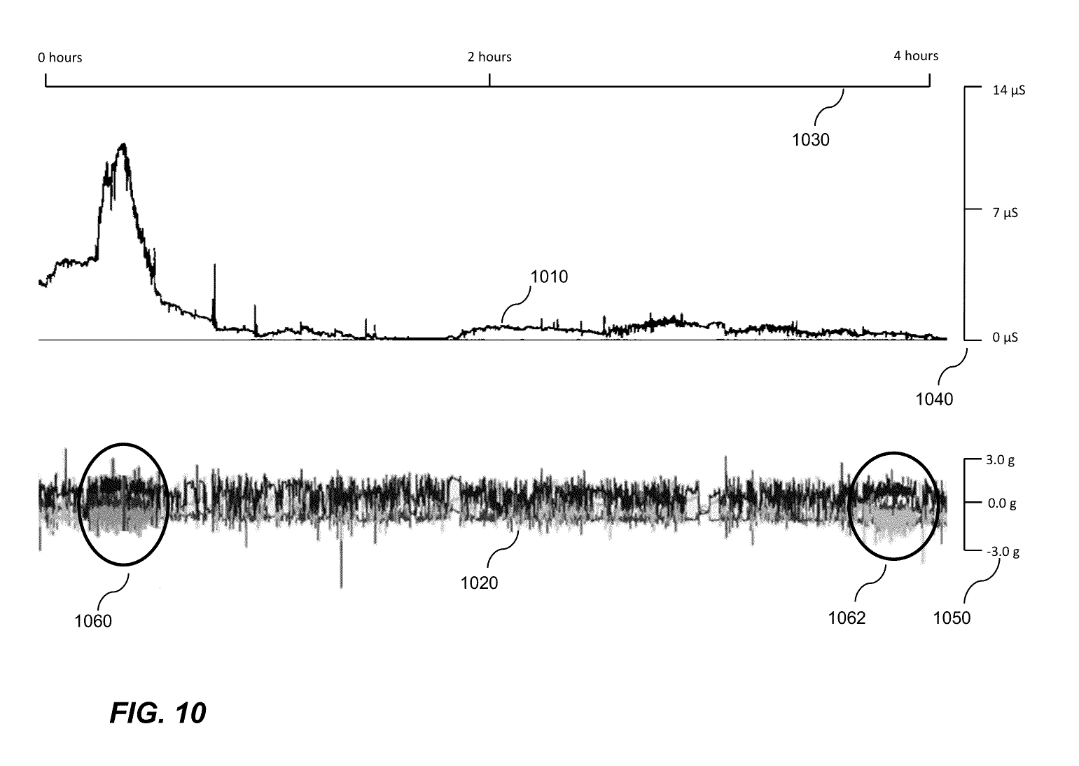

FIG. 10 is a graphical rendering of example electrodermal activity and accelerometer data. The electrodermal activity data graph 1010 is shown along a timeline 1030 with values measured in micro-siemens 1040. Also shown is accelerometer data 1020 with three graphs superimposed for x-axis, y-axis, and z-axis motion. The same timeline 1030 is used for the accelerometer data 1020 and the values for the accelerometer data are shown in g-forces 1050. Two specific times are identified with especially high activity in the accelerometer data. The first timeframe of 1060 is shown for high accelerometer data activity that corresponds with an elevated electrodermal activity. The second timeframe 1062 does not have a corresponding elevated electrodermal activity. Instead the electrodermal activity is low. In this example the first timeframe 1060 corresponds to one drug usage while the second timeframe 1062 corresponds to a second drug usage. By using the electrodermal activity and the accelerometer data in tandem, a signature can be developed to identify when a certain drug used. Likewise other physiological conditions can be identified by such signature analysis.

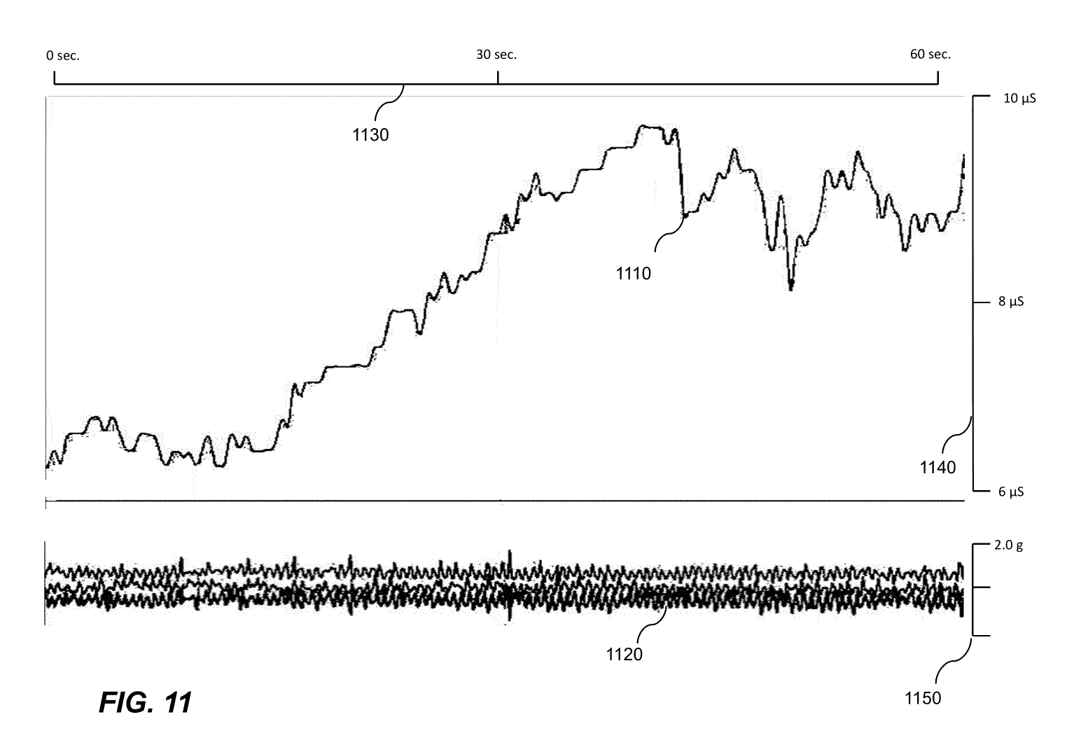

FIG. 11 is a graphical rendering of part of the electrodermal activity and accelerometer data. The graphs shown in FIG. 11 are subsection of the graphs shown in FIG. 10. The graphs in FIG. 11 focus on the timeframe when the electrodermal activity is rising in FIG. 10. The electrodermal activity data graph 1110 is shown along a timeline 1130 with values measured in micro-siemens 1140. Also shown is accelerometer data 1120 with three graphs superimposed for x-axis, y-axis, and z-axis motion. The same timeline 1130 is used for the accelerometer data 1020 and the values for the accelerometer data are shown in g-forces 1150. The accelerometer data can be seen to have a high frequency component with dense oscillations.

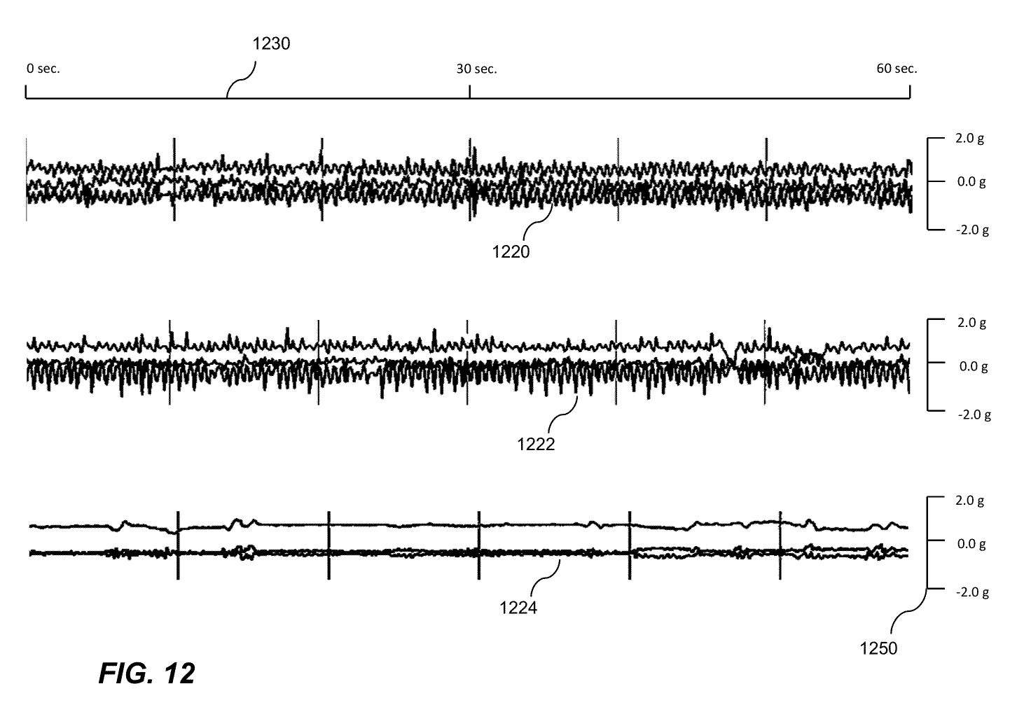

FIG. 12 is a graphical rendering of portions of the accelerometer data. Three different portions of accelerometer data from FIG. 10 are included in FIG. 12 with each portion being expanded. A timeline 1230 is shown having a total duration of 60 seconds. The accelerometer data with three graphs superimposed for x-axis, y-axis, and z-axis motion is shown for each section. The first set of accelerometer data 1220 corresponds to the timeframe 1060 in FIG. 10. The second set of accelerometer data 1222 corresponds to the timeframe 1062 in FIG. 10. The third set of accelerometer data 1224 corresponds to a timeframe in the middle between timeframes 1060 and 1062 in FIG. 10. As can be seen, the accelerometer data 1224 is much quieter than the accelerometer data 1220 and accelerometer data 1224. Accelerometer data 1220 corresponds to the usage of one drug while accelerometer data 1222 corresponds to the usage of another drug. Accelerometer data 1224 corresponds to a timeframe when no drug is being used. Based on this accelerometer data and the electrodermal activity, differences can be determined and a signature can be identified for certain drug usage. Likewise, other physiological conditions can be similarly identified.

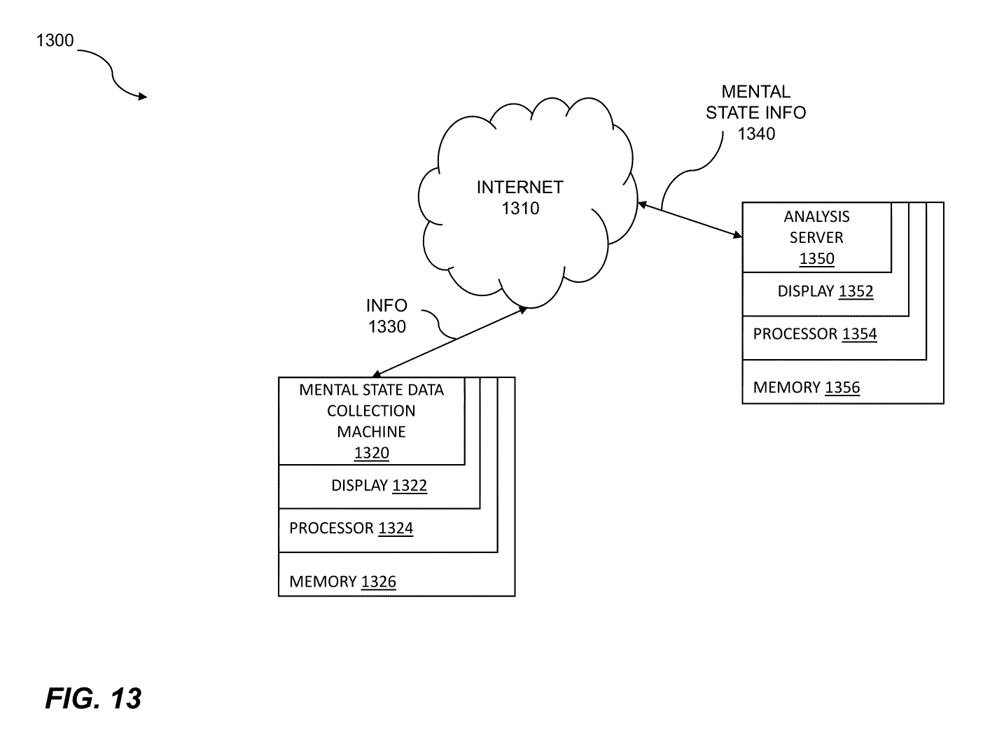

FIG. 13 is a system diagram for mental state analysis. A system 1300 may include a mental state data collection machine 1320 and an analysis server 1350. The mental state data collection machine 1320 and the analysis server 1350 may communicate over the internet 1310 or other computer network.

A mental state data collection machine 1320 has a memory 1326 which stores instructions, and one or more processors 1324 attached to the memory 1326 wherein the one or more processors 1324 can execute instructions stored in the memory 1326. The memory 1326 may be used for storing instructions, for storing mental state data, for system support, and the like. The mental state data collection machine 1320 also may have an Internet connection to carry viewer mental state information 1330, and a display 1322 that may present various advertisements, products, or services to one or more viewers. The client computer 1320 may be able to collect mental state data from one or more people. In some embodiments there may be multiple mental state data collection machines 1320 that each may collect mental state data. The mental state data collection machine 1320 may capture autonomic data which may be used for signature analysis and/or clinical analysis. The autonomic data may include electrodermal activity data. Additionally the mental state data collection machine may capture accelerometer data. The autonomic data, a subset of the autonomic data, or an initial analysis of the autonomic data (performed by the mental state data collection machine 1320) may be communicated as information 1330 across a network and received at the analysis server 1350. The information 1330 may be communicated to the analysis server 1350 without any intervening computation. In some embodiments, the information 1330 may be manipulated and resulting mental state information may be received by the analysis server.

The analysis server 1350 may have a connection to the Internet 1310 to enable mental state information 1340 to be received by the analysis server 1350. Further, the analysis server 1350 may have a memory 1356 which stores instructions, data, mental state data, help information and the like, and one or more processors 1354 coupled to the memory 1356 wherein the one or more processors 1354 can execute instructions. The analysis server 1350 may use its Internet, or other computer communication method, to obtain mental state information 1340. The analysis computer 1350 may receive mental state information collected from a plurality of people from the mental state data collection machine or machines 1320, and may aggregate mental state information on the plurality of people. Analysis may be performed by the analysis server 1350 (web service) through cloud computation of at least one of the autonomic data, a subset of the autonomic data, or an initial analysis of the autonomic data.

The analysis server 1350 may perform a computer-implemented method for physiology analysis comprising: receiving autonomic data on an individual into a web-server computer system wherein the autonomic data provides information for evaluating physiology of the individual and wherein the autonomic data is captured through at least one sensor; analyzing the autonomic data on the individual which was captured; and sending an output related to analyzing that was performed. In at least one embodiment, the mental state data collection machine 1320 and the analysis server 1350 functions are combined into one machine. The system 1300 may include code for capturing autonomic data on an individual into a computer system wherein the autonomic data provides information for evaluating physiology of the individual and wherein the autonomic data is captured through at least one sensor; code for receiving analysis from a web service wherein the analysis is based on the autonomic data on the individual which was captured; and code for rendering an output related to physiology based on the analysis which was received. The system 1300 may include code for capturing electrodermal activity data on an individual into a computer system wherein the electrodermal activity data provides information on physiology of the individual and wherein the electrodermal activity data is captured through a sensor; code for receiving analysis from a web service wherein the analysis is based on the electrodermal activity data on the individual which was captured; and code for rendering an output related to the physiology based on the analysis which was received.

Each of the above methods may be executed on one or more processors on one or more computer systems. Embodiments may include various forms of distributed computing, client/server computing, and cloud based computing. Further, it will be understood that for each flow chart in this disclosure, the depicted steps or boxes are provided for purposes of illustration and explanation only. The steps may be modified, omitted, or re-ordered and other steps may be added without departing from the scope of this disclosure. Further, each step may contain one or more sub-steps. While the foregoing drawings and description set forth functional aspects of the disclosed systems, no particular arrangement of software and/or hardware for implementing these functional aspects should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. Each element of the block diagrams and flowchart illustrations, as well as each respective combination of elements in the block diagrams and flowchart illustrations, illustrates a function, step or group of steps of the methods, apparatus, systems, computer program products and/or computer-implemented methods. Any and all such functions may be implemented by computer program instructions, by special-purpose hardware-based computer systems, by combinations of special purpose hardware and computer instructions, by combinations of general purpose hardware and computer instructions, by a computer system, and so on. Any and all of which may be generally referred to herein as a “circuit,” “module,” or “system.”

A programmable apparatus that executes any of the above mentioned computer program products or computer implemented methods may include one or more processors, microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are not limited to applications involving conventional computer programs or programmable apparatus that run them. It is contemplated, for example, that embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized. The computer readable medium may be a non-transitory computer readable medium for storage. A computer readable storage medium may be electronic, magnetic, optical, electromagnetic, infrared, semiconductor, or any suitable combination of the foregoing. Further computer readable storage medium examples may include an electrical connection having one or more wires, a portable computer diskette, a hard disk, a random access memory (RAM), a read-only memory (ROM), an erasable programmable read-only memory (EPROM), Flash, MRAM, FeRAM, phase change memory, an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed more or less simultaneously to enhance utilization of the processor and to facilitate substantially simultaneous functions. By way of implementation, any and all methods, program codes, program instructions, and the like described herein may be implemented in one or more thread. Each thread may spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the entity causing the step to be performed.

While the invention has been disclosed in connection with preferred embodiments shown and described in detail, various modifications and improvements thereon will become apparent to those skilled in the art. Accordingly, the spirit and scope of the present invention is not to be limited by the foregoing examples, but is to be understood in the broadest sense allowable by law.

1. A computer-implemented method for analyzing physiology comprising:
capturing autonomic data on an individual into a computer system wherein the autonomic data provides information for evaluating physiology of the individual and wherein the autonomic data is captured through at least one sensor;
receiving analysis from a web service wherein the analysis is based on the autonomic data on the individual which was captured; and
rendering an output related to physiology based on the analysis which was received.

2. The method of claim 1 wherein the autonomic data includes electrodermal activity.

3. The method of claim 2 further comprising collecting accelerometer data on the individual.

4. The method of claim 3 further comprising determining contextual information based on one or more of skin temperature or the accelerometer data.

5. The method of claim 4 wherein the contextual information includes information for an activity that the individual is performing.

6. (canceled)

7. The method of claim 1 further comprising uploading the autonomic data to the web service.

8. The method of claim 7 wherein the uploading is accomplished using a mobile device.

9. (canceled)

10. The method of claim 1 further comprising collecting further data on one or more of heart rate, heart rate variability, accelerometer readings, respiration, or skin temperature.

11. The method of claim 1 wherein the sensor is attached to an appendage of the individual.

12. The method of claim 1 wherein the output, which is rendered, describes the physiology of the individual.

13. The method of claim 2 further comprising performing signature analysis on the electrodermal activity which was collected to identify a characteristic of the electrodermal activity that corresponds to a physiological condition.

14. The method of claim 13 wherein the signature analysis is based on the electrodermal activity as well as accelerometer data.

15. The method of claim 13 wherein the signature analysis is based on the electrodermal activity as well as one or more of heart rate, heart rate variability, respiration, or skin temperature.

16. The method of claim 13 further comprising inferring that the individual is experiencing a physiological condition based on observing the characteristic of the autonomic data.

17. The method of claim 16 wherein the inferring includes a probability factor that the person is experiencing the physiological condition.

18. The method of claim 16 wherein the physiological condition that is being experienced is one or more of a group including pain, anxiety, panic attack, epileptic seizure, sleep disorder, respiratory sleep problem, heart attack, depression, stress, reaction to medication, bipolar attack, distracted driving, concussion, stroke, autistic reaction, ADHD behavior, boredom, well being, being startled, being in a mood, having arthritis, cystic fibrosis, diabetes, addiction, eczema outbreak, fragile X syndrome reaction, obsessive-compulsive disorder, phobia, post-traumatic stress disorder, social anxiety disorder, or activation of sympathetic nervous system.

19. The method of claim 13 wherein the characteristic is used to provide a biomarker.

20. The method of claim 13 wherein the signature analysis includes rise time analysis of the electrodermal activity from onset of the physiological condition.

21. The method of claim 13 wherein the signature analysis includes fall time analysis of the electrodermal activity from a peak value following onset of the physiological condition.

22. The method of claim 13 wherein the signature analysis includes time duration for elevated electrodermal activity following onset of the physiological condition.

23. The method of claim 13 wherein the signature analysis includes analysis of electrodermal activity peak to electrodermal activity valley range.

24. The method of claim 13 wherein the signature analysis includes an evaluation of a number of electrodermal activity peaks per minute.

25. The method of claim 13 wherein the signature analysis includes evaluation of electrodermal activity storming.

26. The method of claim 13 further comprising correlating the signature analysis to self-reporting.

27. The method of claim 2 wherein the electrodermal activity is collected with a plurality of sensors on the individual and the plurality of sensors includes at least one sensor on a left side of the individual and at least one sensor on a right side of the individual.

28. The method of claim 27 wherein the at least one sensor on the left side and the at least one sensor on the right side are used to identify a difference between dominant and non-dominant electrodermal activity which was collected.

29. The method of claim 1 wherein the physiological condition is analyzed as part of a clinical trial.

30. The method of claim 1 further comprising capturing autonomic data on a plurality of other people wherein the autonomic data is captured through one or more sensors.

31-34. (canceled)

35. The method of claim 30 further comprising one or more of detecting, clustering, and characterizing patterns in the autonomic data which was captured.

36. (canceled)

37. The method of claim 1 further comprising generating the analysis by the web service through cloud computation of at least one of the autonomic data, a subset of the autonomic data, or an initial analysis of the autonomic data.

38-39. (canceled)

40. The method of claim 13 further comprising, based on a signature, performing one or more of identifying when a treatment was not taken, determining treatment efficacy, analyzing time of day for treatment, identifying an adverse reaction to treatment, identifying side effects to treatment, determining treatment safety, or determining dose titration.

41-44. (canceled)

45. A computer-implemented method for physiology analysis comprising:
receiving autonomic data on an individual into a web-server computer system wherein the autonomic data provides information for evaluating physiology of the individual and wherein the autonomic data is captured through at least one sensor;
analyzing the autonomic data on the individual which was captured; and
sending an output related to analyzing that was performed.

46. A computer program product embodied in a non-transitory computer readable medium for physiology analysis, the computer program product comprising:
code for capturing autonomic data on an individual into a computer system wherein the autonomic data provides information for evaluating physiology of the individual and wherein the autonomic data is captured through at least one sensor;
code for receiving analysis from a web service wherein the analysis is based on the autonomic data on the individual which was captured; and
code for rendering an output related to physiology based on the analysis which was received.

47. A computer system for physiology analysis comprising:
a memory which stores instructions;
one or more processors coupled to the memory wherein the one or more processors, when executing the instructions which are stored, are configured to:
capture autonomic data on an individual into a computer system wherein the autonomic data provides information for evaluating physiology of the individual and wherein the autonomic data is captured through at least one sensor;
receive analysis from a web service wherein the analysis is based on the autonomic data on the individual which was captured; and
render an output related to physiology based on the analysis which was received.
